SEQUENCE LISTING (11 sequences)

SEQ ID NO: 1 - DNA, 1230 bp, synthetic construct
  misc_feature 1..1230: note = The sequence is synthetized.
  source 1..1230: mol_type = other DNA
atggttcgttacgtgccccggttcgctgatggtcagaaagtaaagttggcttggcccttggcggtttttcggttaaatcacatattctggccattggatccgagcacagggaaatggggccgatatctggacaaggttctagctgttgcgatgtccttggtttttatgcaacacaacgatgcagagctgaggtacttgcgcttcgaggcaagtaatcggaatttggatgcctttctcacaggaatgccaacgtatttaatcctcgtggaggctcaatttagaagtcttcacattctactgcacttcgagaagcttcagaagtttttagaaatattctacgcaaatatttatattgatccccgtaaggaacccgaaatgtttcgaaaagtggatggaaagatgataattaacagattagtttcggccatgtacggtgcagttatctctctgtatctaatcgcacccgttttttccatcattaaccaaagcaaagattttctatactctatgatctttccgttcgattcggatcccttgtacatatttgtgccactgcttttgacaaacgtatgggttggcattgtaatagataccatgatgttcggggagacgaatttgttgtgtgaactaattgtccacctaaatggtagttatatgttgctcaagagggacttgcagttggccattgaaaagatattagttgcaagggaccgtccgcatatggccaaacagctaaaggttttaattacaaaaactctccgaaagaatgtggctctaaatcagtttggccagcagctggaggctcagtatactgtgcgggtttttattatgtttgcattcgctgcgggccttttatgtgctctttcttttaaggcttatacgaatcctatggctaattacatctatgcgatttggtttggtgctaagacagttgagctgctttctttaggacagattggttccgacttggcctttactacggattccctcagcacaatgtactaccttacccattgggagcaaatcctgcagtactctacaaatcccagcgaaaatctgcgattactaaagctcattaacttggccattgagatgaacagcaagcccttctatgtgacagggctaaaatattttcgcgttagtctgcaggctggcttaaaaattctgcaggcatccttctcgtacttcacattcctcacttcgatgcagcgacgacaaatgagcaattaa

SEQ ID NO: 2 - DNA, 1461 bp, synthetic construct
  misc_feature 1..1461: note = The sequence is synthetized.
  source 1..1461: mol_type = other DNA
atgacaacctcgatgcagccgagcaagtacacgggcctggtcgccgacctgatgcccaacatccgggcgatgaagtactccggcctgttcatgcacaacttcacgggcggcagtgccttcatgaagaaggtgtactcctccgtgcacctggtgttcctcctcatgcagttcaccttcatcctggtcaacatggccctgaacgccgaggaggtcaacgagctgtcgggcaacacgatcacgaccctcttcttcacccactgcatcacgaagtttatctacctggctgttaaccagaagaatttctacagaacattgaatatatggaaccaggtgaacacgcatcccttgttcgccgagtcggatgctcgttaccattcgatcgcactggcgaagatgaggaagctgttctttctggtgatgctgaccacagtcgcctcggccaccgcctggaccacgatcaccttctttggcgacagcgtaaaaatggtggtggaccatgagacgaactccagcatcccggtggagataccccggctgccgattaagtccttctacccgtggaacgccagccacggcatgttctacatgatcagctttgcctttcagatctactacgtgctcttctcgatgatccactccaatctatgcgacgtgatgttctgctcttggctgatattcgcctgcgagcagctgcagcacttgaagggcatcatgaagccgctgatggagctgtccgcctcgctggacacctacaggcccaactcggcggccctcttcaggtccctgtcggccaactccaagtcggagctaattcataatgaagaaaaggatcccggcaccgacatggacatgtcgggcatctacagctcgaaagcggattggggcgctcagtttcgagcaccctcgacactgcagtcctttggcgggaacgggggcggaggcaacgggttggtgaacggcgctaatcccaacgggctgaccaaaaagcaggagatgatggtgcgcagtgccatcaagtactgggtcgagcggcacaagcacgtggtgcgactggtggctgccatcggcgatacttacggagccgccctcctcctccacatgctgacctcgaccatcaagctgaccctgctggcataccaggccaccaaaatcaacggagtgaatgtctacgccttcacagtcgtcggatacctaggatacgcgctggcccaggtgttccacttttgcatctttggcaatcgtctgattgaagagagttcatccgtcatggaggccgcctactcgtgccactggtacgatggctccgaggaggccaagaccttcgtccagatcgtgtgccagcagtgccagaaggcgatgagcatatcgggagcgaaattcttcaccgtctccctggatttgtttgcttcggttctgggtgccgtcgtcacctactttatggtgctggtgcagctcaagtaa

SEQ ID NO: 3 - protein, 933 aa, synthetic construct
  REGION 1..933: note = The sequence is synthetized.
MVRYVPRFADGQKVKLAWPLAVFRLNHIFWPLDPSTGKWGRYLDKVLAVAMSLVFMQHNDAELRYLRFEASNRNLDAFLTGMPTYLILVEAQFRSLHILLHFEKLQKFLEIFYANIYIDPRKEPEMFRKVDGKMIINRLVSAMYGAVISLYLIAPVFSIINQSKDFLYSMIFPFDSDPLYIFVPLLLTNVWVGIVIDTMMFGETNLLCELIVHLNGSYMLLKRDLQLAIEKILVARDRPHMAKQLKVLITKTLRKNVALNQFGQQLEAQYTVRVFIMFAFAAGLLCALSFKAYTNPMANYIYAIWFGAKTVELLSLGQIGSDLAFTTDSLSTMYYLTHWEQILQYSTNPSENLRLLKLINLAIEMNSKPFYVTGLKYFRVSLQAGLKILQASFSYFTFLTSMQRRQMSNATNFSLLKQAGDVEENPGPMTTSMQPSKYTGLVADLMPNIRAMKYSGLFMHNFTGGSAFMKKVYSSVHLVFLLMQFTFILVNMALNAEEVNELSGNTITTLFFTHCITKFIYLAVNQKNFYRTLNIWNQVNTHPLFAESDARYHSIALAKMRKLFFLVMLTTVASATAWTTITFFGDSVKMVVDHETNSSIPVEIPRLPIKSFYPWNASHGMFYMISFAFQIYYVLFSMIHSNLCDVMFCSWLIFACEQLQHLKGIMKPLMELSASLDTYRPNSAALFRSLSANSKSELIHNEEKDPGTDMDMSGIYSSKADWGAQFRAPSTLQSFGGNGGGGNGLVNGANPNGLTKKQEMMVRSAIKYWVERHKHVVRLVAAIGDTYGAALLLHMLTSTIKLTLLAYQATKINGVNVYAFTVVGYLGYALAQVFHFCIFGNRLIEESSSVMEAAYSCHWYDGSEEAKTFVQIVCQQCQKAMSISGAKFFTVSLDLFASVLGAVVTYFMVLVQLKATNFSLLKQAGDVEENPGP

SEQ ID NO: 4 - protein, 409 aa, synthetic construct
  REGION 1..409: note = The sequence is synthetized.
MVRYVPRFADGQKVKLAWPLAVFRLNHIFWPLDPSTGKWGRYLDKVLAVAMSLVFMQHNDAELRYLRFEASNRNLDAFLTGMPTYLILVEAQFRSLHILLHFEKLQKFLEIFYANIYIDPRKEPEMFRKVDGKMIINRLVSAMYGAVISLYLIAPVFSIINQSKDFLYSMIFPFDSDPLYIFVPLLLTNVWVGIVIDTMMFGETNLLCELIVHLNGSYMLLKRDLQLAIEKILVARDRPHMAKQLKVLITKTLRKNVALNQFGQQLEAQYTVRVFIMFAFAAGLLCALSFKAYTNPMANYIYAIWFGAKTVELLSLGQIGSDLAFTTDSLSTMYYLTHWEQILQYSTNPSENLRLLKLINLAIEMNSKPFYVTGLKYFRVSLQAGLKILQASFSYFTFLTSMQRRQMSN

SEQ ID NO: 5 - protein, 486 aa, synthetic construct
  REGION 1..486: note = The sequence is synthetized.
MTTSMQPSKYTGLVADLMPNIRAMKYSGLFMHNFTGGSAFMKKVYSSVHLVFLLMQFTFILVNMALNAEEVNELSGNTITTLFFTHCITKFIYLAVNQKNFYRTLNIWNQVNTHPLFAESDARYHSIALAKMRKLFFLVMLTTVASATAWTTITFFGDSVKMVVDHETNSSIPVEIPRLPIKSFYPWNASHGMFYMISFAFQIYYVLFSMIHSNLCDVMFCSWLIFACEQLQHLKGIMKPLMELSASLDTYRPNSAALFRSLSANSKSELIHNEEKDPGTDMDMSGIYSSKADWGAQFRAPSTLQSFGGNGGGGNGLVNGANPNGLTKKQEMMVRSAIKYWVERHKHVVRLVAAIGDTYGAALLLHMLTSTIKLTLLAYQATKINGVNVYAFTVVGYLGYALAQVFHFCIFGNRLIEESSSVMEAAYSCHWYDGSEEAKTFVQIVCQQCQKAMSISGAKFFTVSLDLFASVLGAVVTYFMVLVQLK

SEQ ID NO: 6 - DNA, 27 bp, synthetic construct
  misc_feature 1..27: note = The sequence is synthetized.
  source 1..27: mol_type = other DNA
gctctagaatggttcgttacgtgcccc

SEQ ID NO: 7 - DNA, 78 bp, synthetic construct
  misc_feature 1..78: note = The sequence is synthetized.
  source 1..78: mol_type = other DNA
aggtccagggttctcctccacgtctccagcctgcttcagcaggctgaagttagtagcattgctcatttgtcgtcgctg

SEQ ID NO: 8 - DNA, 77 bp, synthetic construct
  misc_feature 1..77: note = The sequence is synthetized.
  source 1..77: mol_type = other DNA
gctactaacttcagcctgctgaagcaggctggagacgtggaggagaaccctggacctatgacaacctcgatgcagcc

SEQ ID NO: 9 - DNA, 83 bp, synthetic construct
  misc_feature 1..83: note = The sequence is synthetized.
  source 1..83: mol_type = other DNA
ccaccggtaggtccagggttctcctccacgtctccagcctgcttcagcaggctgaagttagtagccttgagctgcaccagcac

SEQ ID NO: 10 - DNA, 27 bp, synthetic construct
  misc_feature 1..27: note = The sequence is synthetized.
  source 1..27: mol_type = other DNA
gctctagaatggttcgttacgtgcccc

SEQ ID NO: 11 - DNA, 83 bp, synthetic construct; identical to SEQ ID NO: 9
  misc_feature 1..83: note = The sequence is synthetized.
  source 1..83: mol_type = other DNA

(19) United States
(12) Patent Application Publication
(10) Pub. No.: US 2026/0297006 A1
(43) Pub. Date: Oct. 1, 2026

(54) USE OF 2-PENTANONE AND SPECIFIC RECEPTOR THEREOF IN MANUFACTURE OF PRODUCTS REGULATING CELL FUNCTIONS

(71) Applicant: Creator (Wuhan) Medicine Technology Co., Ltd., Wuhan (CN)

(72) Inventors: Changbin KE, Wuhan (CN); Yanqiong WU, Wuhan (CN); Shanchun SU, Wuhan (CN); Xueqin XU, Wuhan (CN)

(21) Appl. No.: 19/676,005

(22) Filed: May 13, 2026

Related U.S. Application Data
(62) Division of application No. 17/817,010, filed on Aug. 3, 2022, now abandoned.

(30) Foreign Application Priority Data
Jan. 27, 2022 (CN) 202210102995.7

(51) Int. Cl. C07C 49/20

(52) U.S. Cl. CPC C07C 49/20; C12N 2502/00

(57) ABSTRACT

A method for regulating a cell includes expressing a specific receptor of 2-pentanone in a target cell of the specific receptor, wherein the target cell is regulated by a binding of the 2-pentanone to the specific receptor of the 2-pentanone to activate a cyclic ligand-gated cation channel and result in the increase in the intracellular calcium ion concentration, a depolarization of cell membranes, and a generation of an electrical activity or a corresponding physiological activity. The specific receptor of 2-pentanone is expressed in cultured cells or animals, and its specific binding to 2-pentanone opens ligand-gated cation channels, resulting in an increase of intracellular calcium ion concentration, depolarization of cell membranes, and generation of electrical activity or endocrine activity, thereby finally achieving precise regulation of tissue cells and organ functions.

### CROSS REFERENCE TO THE RELATED APPLICATIONS

This application is a Divisional of co-pending application Ser. No. 17/817,010, filed on Aug. 3, 2022 for which priority is claimed under 35 U.S.C. § 120; and this application claims priority to Chinese Patent Application No: 202210102995.7, filed on Jan. 27, 2022, under 35 U.S.C. § 119 (e), the entire contents of which are incorporated herein by reference.

### REFERENCE TO AN ELECTRONIC SEQUENCE LISTING

The content of the electronic sequence listing (SEQLXML26_sequence_lisiting.xml; Size: 19,576 bytes; and Date of Creation: May 12, 2026) is herein incorporated by reference in its entirety.

### TECHNICAL FIELD

The present disclosure relates to the field of biotechnology, and specifically to use of 2-pentanone and specific receptor thereof in the manufacture of products regulating cell functions.

### BACKGROUND

In the field of fundamental life science study, neurons, cardiomyocytes and skeletal muscle cells are able to release or transmit action potentials, and thus have important physiological functions in life processes. For in-depth studies, researchers usually activate neurons in a particular area of the brain, or simulate cardiomyocytes, skeletal muscle cells and intracellular calcium-dependent physiological functions (e.g., cell secretion), by some means, to activate certain particular behavior or to produce some important physiological functions. Application of such techniques plays an important role in the treatment of diseases including mental diseases, neurological disorders, myocardial dysfunction, motor dysfunction and endocrine disorders.

Optogenetics and chemogenetics have been commonly used in these field. Optogenetics requires cells in a target area to express light-sensitive channel proteins such as ChR2 and NpHR, and an optical fiber connected to an external laser is embedded in this area, so that the laser beam with a specific wavelength activates the corresponding light-sensitive channel proteins in that area, so as to excite or inhibit the cells in that area. Optogenetics is advantaged in that it precisely controls the treatment time or intensity and exhibits high efficiency and stable effects, owing to using laser. The treatment based on optogenetics, however, cannot be carried out for a long time, due to heat generation from laser beams. In addition, the optogenetics requires accessories such as optical fibers, laser controllers and catheters in its use, and to embed optical fibers is delicate and complicated, which cause difficulty in operation and many complications. Moreover, heads of the optical fibers need to be fixed inside the cranium of animals during the later experiments, which brings inconvenience to them and also injury to cells by laser itself. Therefore, it is inconvenient to clinically apply optogenetics.

Chemogenetics requires cells in a target area to express artificially engineered chemoreceptors such as hM3Dq and hM4Di, in which the receptors expressed by the cells in the target area are activated by feeding or injecting agonists (Clozapine-N-Oxide, CNO) of these receptors into the body, to excite or inhibit activity of the cells. Chemogenetics needs less instruments, can be simply operated in experiments and processed for a long time, and thus has a promising clinical application. However, chemogenetics takes effect very slowly (more than 0.5 hours), failing to control time and intensity accurately, and failing to work rapidly and to be eluted out rapidly, due to metabolic properties of CNO in the body.

In summary, the conventional optogenitics and chemogenetics are disadvantaged in controlling the activity of cells, since they are not suitable for long-term use, complicated in operations, inconvenient, and lacking of accuracy, and also cause difficult for cells to recover.

### SUMMARY

In view of the forgoing, embodiments of the present disclosure provide a 2-pentanone receptor and use thereof, so as to solve the problems, of the conventional optogenitics and chemogenetics for controlling the physiological activities of cells, that they are not suitable for long-term use, complicated in operations, inconvenient, and lacking of accuracy.

In order to achieve the above object, embodiments of the present disclosure provides the following technical solutions:

According to embodiments of the present disclosure, use of 2-pentanone and specific receptor thereof in (A1) manufacture of a product regulating cell function, (A2) regulation of cell function, (A3) manufacture of a product promoting an increase in intracellular calcium ion concentration, or (A4) manufacture of a product promoting an increase in neuronal firing rate, is provided.

In an embodiment of the present disclosure, the specific receptor of 2-pentanone comprises Or35a having an amino acid sequence set forth in SEQ ID NO: 4 and Or83b having an amino acid sequence set forth in SEQ ID NO: 5.

In an embodiment of the present disclosure, the gene encoding the specific receptor Or35a has a nucleotide sequence set forth in SEQ ID NO: 1, and the gene encoding the specific receptor Or83b has a nucleotide sequence set forth in SEQ ID NO: 2.

An expression vector, recombinant vector or recombinant tool cell comprising the gene encoding the specific receptor described above is also within the scope of the present invention.

In an embodiment of the present disclosure, the recombinant vector is an Or35a-P2A-Or83b-P2A-GV320 expression plasmid vector. In an embodiment of the present disclosure, the product is a pharmaceutical product or a reagent. The present disclosure also provides a method for regulating a cell, comprising expressing the specific receptor as described above in a target cell of the receptor, wherein the target cell is regulated by the binding of 2-pentanone to the specific receptor of 2-pentanone which activates a cyclic ligand-gated cation channel and results in an increase in intracellular calcium ion concentration, depolarization of cell membranes, and generation of an electrical activity or a corresponding physiological activity.

In an embodiment of the present disclosure, the specific receptor of 2-pentanone is expressed in the target cell via a recombinant expression vector or recombinant cell comprising the gene encoding 2-pentanone-receptors. The expression sequence can be added with certain modification elements (such as Cre-loxp regulatory elements) at its upstream and downstream to optimize or regulate its expression efficiency or function. The modified sequence also falls within the scope of the claims of this application, since it would still work to specifically express the receptor.

According to the present disclosure, the specific binding of 2-pentanone to the specific receptor of 2-pentanone will open ligand-gated cation channels, resulting in an increase of intracellular calcium ion concentration and thus precise regulation of cell or tissue organ functions by regulating physiological activities such as electrical activity or endocrine of cells. According to the embodiments of the present disclosure, 2-pentanone and specific receptor thereof are utilized as a basis to specifically regulate physiological activity of cells. 2-Pentanone, being as an odorant molecule with good volatile properties and a certain degree of water solubility, can cross the blood-brain barrier, and thus enter the blood system and central nervous system through inhalation to reach all parts of the body. Odor receptors are mainly expressed on olfactory cells of olfactory epithelium and can be specifically activated by corresponding odorant molecules. Odor receptors belong to a class of G-protein coupled receptors. Specific odorant receptors for 2-pentanone include Or35a, Or83b and the like. The specific receptor of 2-pentanone is ectopically expressed in a specific area of a mice in advance, and the receptor-expressing cells are efficiently activated by taking advantage that 2-pentanone can rapidly reach various parts of the body through inhalation, thus producing an effect. According to the present disclosure, in addition to the specific receptor, amino acid sequences including Igk are also expressed in the target area to assist in the transport of the receptor of 2-pentanone to cell membranes and to form a functional structure.

Embodiments of the present disclosure have the following advantages:

According to embodiment of the present disclosure, the specific receptor of 2-pentanone is expressed in cultured cells or animals, and its specific binding to 2-pentanone opens ligand-gated cation channels, resulting in an increase of intracellular calcium ion concentration, depolarization of cell membranes, and generation of electrical activity or endocrine activity. The experimental operation is simple and does not require too many instruments.

When being used in treatment, 2-pentanone and the specific receptor of 2-pentanone according to the present disclosure can activate cells rapidly, producing effects with a rapid onset; once the treatment is stopped, the experimental effect can be quickly terminated to allow the cells to return to their original state quickly; the treatment can be performed for a long time, since it does not harm the cells, even in the case of a prolonged treatment; and thus they can be easily applied clinically to treat diseases.

The technique according to the present disclosure can be operated simply, rapidly produce effects which also can be terminated rapidly, and is reusable, so it is promising to be applied clinically.

### BRIEF DESCRIPTION OF THE DRAWINGS

Hereinafter, to more clearly illustrate the technical solutions in the embodiments of the present disclosure or the existing technology, a brief introduction will be made to the drawings necessary to illustrate the embodiments or the existing technology. But, it is obvious that the drawings below are only exemplary and that it is possible for these skilled in the art to derive other implementation drawings from them without any inventive effort.

FIG. 1 is a schematic diagram showing use of 2-pentanone and a 2-pentanone receptor according to an embodiment of the present disclosure;

FIG. 2 is a schematic diagram of an Or35a-P2A-Or83b-P2A-GV320 expression plasmid vector constructed according to an embodiment of the present disclosure;

FIG. 3 is a graph showing the results of detecting 2-pentanone by HPLC-MS according to an embodiment of the present disclosure;

FIG. 4A is a graph showing the results of calcium imaging experiment after the expression of the specific receptor of 2-pentanone in cells according to an embodiment of the present disclosure;

FIG. 4B is a graph showing the results of patch clamp experiment after the expression of the specific receptor of 2-pentanone in cells according to an embodiment of the present disclosure; and

FIG. 5 is a graph showing the results of PCR and Western blot identification of tissues from the F0 generation mouse in an embodiment of the present disclosure.

### DETAILED DESCRIPTION

The embodiments of the present disclosure will be illustrated below using specific examples. Those familiar with this technology can easily understand other advantages and effects of the present disclosure from the contents disclosed in this specification. It is apparent that the described embodiments are only some rather than all of the embodiments of the present disclosure. Any other embodiments obtained by those skilled in the art based on the embodiments in the present disclosure without any creative work fall in the protection scope of the present invention.

### Example 1. Construction of Expression Vectors of the 2-Pentanone Specific Receptor Or35a and Specific Receptor Or83b

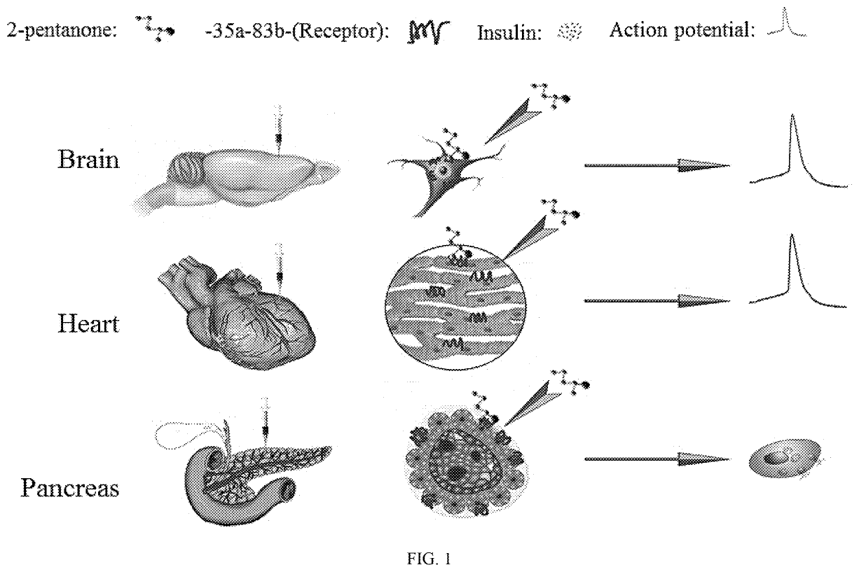

As shown in FIG. 1, the specific receptor of 2-pentanone according to the present disclosure can be expressed in a target area of an animal via a viral vector or by the transgenic technology. When reaching cells in the receptor-expressing area through inhalation and binding to the receptor, 2-pentanone opens ligand-gated cation channels, which results in an increase in intracellular calcium ion concentration, depolarization of cell membranes, generation of electrical activity or endocrine activity, and the other activities.

The expression vectors of the 2-pentanone specific receptor Or35a and specific receptor Or83b in this example were constructed by the following steps:

### I. Construction of Or35a-P2A-Or83b-P2A Co-Expression Gene

1. The extracted cDNA of Drosophila olfactory bulb was used as the template. The nucleotide sequence of the gene of the specific receptor Or35a is shown in SEQ ID NO: 1, and the nucleotide sequence of the gene of the specific receptor Or83b is shown in SEQ ID NO: 2. The amino acid sequence of the protein encoded by the co-expressed sequence of Or35a-P2A-Or83b-P2A gene is shown in SEQ ID NO: 3, the amino acid sequence of the specific receptor 35a is shown in SEQ ID NO: 4, and the amino acid sequence of the specific receptor Or83b is shown in SEQ ID NO: 5. Primers are respectively designed for PCR of Or35a-P2A and Or83b-P2A. Note that the target sequence may also be obtained by direct manual nucleotide-by-nucleotide synthesis according to the sequence.

The primer sequences for amplifying the gene of the receptor Or35a are:

Or35a-P2A-F:
(XbaI)GCTCTAGAATGGTTCGTTACGTGCCCC,
as shown in SEQ ID NO: 6;
Or35a-P2A-R:
AGGTCCAGGGTTCTCCTCCACGTCTCCAGCCTGCTTCAGCAGGCTG
AAGTTAGTAGCATTGCTCATTTGTCGTCGCTG,
as shown in SEQ ID NO: 7;

The primer sequences for amplifying the gene of the receptor Or83b are:

Or83b-P2A-F:
GCTACTAACTTCAGCCTGCTGAAGCAGGCTGGAGACGTGGAGGAGA
ACCCTGGACCTATGACAACCTCGATGCAGCC,
as shown in SEQ ID NO: 8;
Or83b-P2A-R:
(AgeI)CCACCGGTAGGTCCAGGGTTCTCCTCCACGTCTCCAGCCT
GCTTCAGVCAGGCTGAAGTTAGTAGCCTTGAGCTGCACCAGCAC,
as shown in SEQ ID NO: 9;

Reaction system of PCR amplification included ddH₂O to 50 μl, 25 μl of 2*Phanta Max Master Mix, 2 μl of cDNA, 2 μl of an upstream primer (10 μM), and 2 μl of a downstream primer (10 μM). PCR amplification conditions: 1. Pre-denaturation at 95° C. for 3 min; 2. Denaturation at 95° C. for 15 sec; 3. Annealing at 55-60° C. for 15 sec; 4. Extension at 72° C. and 1 min/kb; 5. Final extension at 72° C. for 5 min; 6. 4° C.+∞ at a total of 30-35 cycles.
2. Purification of PCR products: The above PCR products were purified by using the kit (EasyPure PCR Purification Kit, Cat: EP101-02, Beijing TransGen Biotechnology Co. Ltd), and named as product A and product B, respectively. The product A and product B were used as templates to amplify the gene fragment of the co-expressed gene Or35a-P2A-Or83b-P2A using the Overlay PCR method and the following primer sequences.

Or35a-P2A-Or83b-P2A-F:
GCTCTAGAATGGTTCGTTACGTGCCCC(Xbal),
as shown in SEQ ID NO: 10
Or35a-P2A-Or83b-P2A-R:
CCACCGGTAGGTCCAGGGTTCTCCTCCACGTCTCCAGCCTG
CTTCAGCAGGCTGAAGTTAGTAGCCTTGAGCTGCACCAGCA
C(AgeI),
as shown in SEQ ID NO: 11

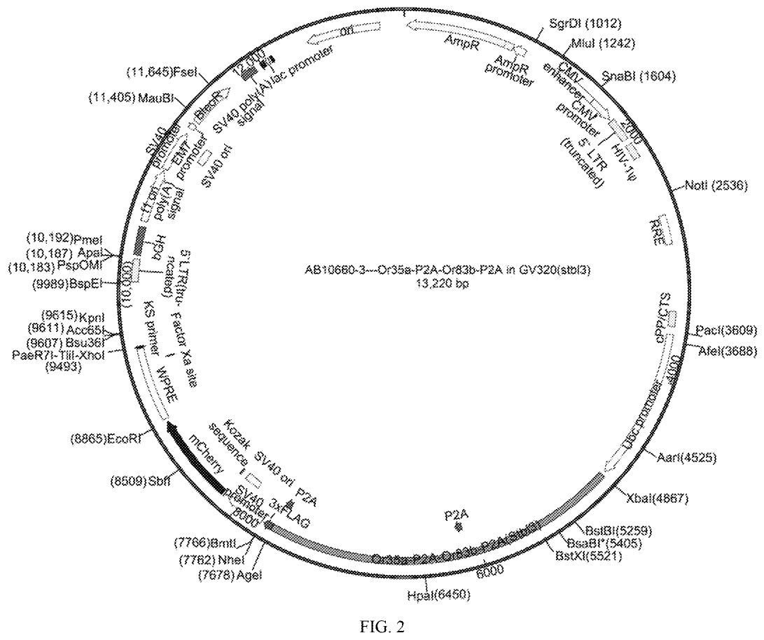

The reaction system included ddH₂O to 50 μl, 25 μl of 2× Phanta Max Master Mix, 2 μl of each of Product A and Product B, 2 μl of an upstream primer (10 μM), and 2 μl of a downstream primer (10 μM). PCR amplification conditions: 1. Pre-denaturation at 95° C. for 3 min; 2. Denaturation at 95° C. for 15 sec; 3. Annealing at 67° C. for 15 sec; 4. Extension at 72° C. for 3 min; decrease by 0.5° C. per cycle; 5. Denaturation at 95° C. for 15 sec; 6. Annealing at 57° C. for 15 sec; 7. Extension at 72° C. for 3 min; 8. Final extension at 72° C. for 5 min; 9.4° C.+co. Reagents: vazyme 2× Phanta Max Master Mix, Cat: P515-03.
3. The Or35a-P2A-Or83b-P2A gene fragment and the GV320 plasmid vector were digested by XbaI and AgeI, ligated by T4 ligase at a certain ratio, and then incubated at 16° C. overnight to generate the ligation product of the Or35a-P2A-Or83b-P2A gene fragment and the GV320 plasmid vector.
4. The ligation product of the Or35a-P2A-Or83b-P2A gene fragment and the GV320 plasmid vector were transformed into E. coli Stb13 competent cells, and the plate was inverted at 37° C. overnight.
5. Single colonies were picked, inoculated in LB (Amp+) liquid medium and cultured overnight with shaking at 37° C. Plasmid extraction was then performed on the bacterial solution using a kit.
6. The extracted plasmids were preliminarily identified by enzyme digestion and sent for sequencing together with designed primers. The plasmid with correct sequencing was the Or35a-P2A-Or83b-P2A-GV320 expression plasmid vector. The constructed Or35a-P2A-Or83b-P2A-GV320 expression plasmid vector is shown in FIG. 2.

### II. Viral Packaging of the Expression Plasmid Vectors

1. HEK 293T cells in good cell condition and endotoxin-free large-scale-extracted recombinant virus core and helper plasmids were prepared. The backbone plasmid Or35a-P2A-Or83b-P2A-GV320 and the shuttle plasmids (pLP1, pLP2 and pLP/VSVG combined) were 16 μg each.
2. Twenty-four hours before transfection, the cells were plated and cultured in a 37° C., 5% CO₂ incubator. After 24 h, HEK293T cells were used for transfection when the cell density reached 70%-80%.
3. HEK293T cell culture medium was changed to serum-free medium 2 h before transfection.
4. The recombinant virus core plasmid and helper packaging plasmid were transfected with the corresponding transfection reagents in a certain ratio. First, 32 μg of the viral vector plasmid to be transfected was dissolved in Opti-MEM medium to a total volume of 500 μl, gently mixed and allowed to stand for 5 min. Secondly, the transfection reagent PEI was dissolved in Opti-MEM medium in a ratio of 1:3 with a total volume of 500 μl, gently mixed and allowed to stand for 5 min. Next, the diluted transfection reagent was added dropwise into the plasmid diluent, mixed gently during the addition, and incubated at room temperature for 20 min to allow the DNA and transfection reagent to fully combine to form a stable transfection complex. Then, the cell culture plate was taken out, added with the prepared DNA-PEI complex and placed back to the incubator for further culture. Finally, 3-5 h after transfection, ½ volume of growth medium containing 30% serum was added for further culture, and after 12 h, the transfection medium was removed and replaced with 10 ml of fresh complete medium.
5. After 48 h of transfection, the cell supernatant enriched with lentiviral particles was collected, concentrated and tested for titer. The lentivirus expressing the specific receptor Or35a and the specific receptor Or83b with titer of ≥1×10⁸ TU/mL was obtained.

In this example, expression elements were ectopically expressed in cultured cells or in vivo using plasmid/viral vector or transgenic technology to generate functional Or35a and Or83b co-expressed receptors, i.e., lentivirus expressing the specific receptor Or35a and the specific receptor Or83b. The binding of 2-pentanone to Or35a and Or83b receptors on the cell surface opens ligand-gated cation channels, resulting in an increase of intracellular calcium ion concentration, depolarization of cell membranes, generation of electrical activity or endocrine activity, and other activities.

### Example 2. Construction of Transgenic Model Animals

In this example, the construction process of transgenic model animals is provided as follows:
1. Construction of homologous recombination vectors: A homologous recombination vector was constructed via CRISPR/Cas9 pathway. The exogenous gene Or35a-P2A-Or83b-P2A fragment was inserted between the homologous arms of Rosa26 gene, and the strong CAG promoter with broad expression profile was selected. After a series of enzyme digestion and identification, the vector with 100% homology verified by sequencing was considered as successfully constructed targeting vector.

Construction and identification of SgRNA vector: (1) gRNA primers were synthesized according to Rosa26 site sequence; (2) Single-stranded oligo was phosphorylated and annealed to form a double strand, which was denatured at 98° C. and then cooled to room temperature to form a DNA fragment with a sticky end; (3) Plasmid PX459 was cut with Bbs I, recovered after electrophoresis and ligated with annealing primers. (4) The ligation product was transformed into competent cells and plated overnight. (5) Single colonies were picked to screen positive clones by colony PCR; (6) The identified positive clones were verified by sequencing; (7) The correct sequencing results indicate that the CRISPR-SgRNA vector was successfully constructed.

Construction of CRISPR/Cas9 vector: The target Cas9 fragment was amplified by PCR and the recovered PCR product was ligated into Nhe I-digested oriP-EBNA 1 vector. The ligation product was transformed into DH-5a competent cells at 42° C., and cells were cultured overnight on agarose plates containing ampicillin for screening. Single colonies were picked to extract the plasmid DNA, and the insert sequence was verified by sequencing.

Construction of homologous arm vector: About 1-2 kb of sequences targeting both ends of Rosa26 site were amplified respectively by PCR as homologous arms, and IRES-Or35a-P2A-Or83b-P2A fragment was also amplified. Left Arm, right Arm and IRES-GFP fragments were ligated into oriP-EBNA1 vector by seamless cloning. The ligation product was transformed into DH-5a competent cells at 42° C., and cells were cultured overnight on agarose plates containing ampicillin for screening. Single colonies were picked to extract the plasmid DNA, and the insert sequence was verified by sequencing.
2. Detection of site-specific integration vector-associated cells: Cas9 vector, sgRNA vector and IRES-Or35a-P2A-Or83b-P2A homologous recombination vector were introduced into mouse embryonic fibroblasts (MEF) by the electroporation transfection method. After 48 h of culture, the genome was extracted. Two pairs of primers were used to detect whether the target gene Or35a-P2A-Or83b-P2A was successfully integrated into the cell genome and inserted into the target site as expected. The PCR product was sequenced for verification.
3. The sgRNA vector, IRES-Or35a-P2A-Or83b-P2A homologous recombination targeting vector and Cas9 vector, which were constructed based on the mechanism of CRISPR/Cas9 system, were injected into the pronuclei of mouse fertilized eggs by pronuclear microinjection.
4. The injected fertilized eggs were transferred to surrogate recipients by embryo transfer technology until the mice were born. The process included that female C57BL/6 mice aged 5-6 weeks were selected and injected intraperitoneally with pregnant mare serum gonadotropin (PMSG) at 5 IU/mouse at 1:00-2:00 μm, and injected intraperitoneally with human chorionic gonadotropin (HCG) at 5 IU/mouse after 46-48 h. Then, they were cohabited and mated with fertile male mice, and the female mice with vaginal plug were selected on the next morning to take fertilized eggs.

The mice were sacrificed. A small segment of uterus with intact oviduct was cut off after stripping and placed in M2 culture medium. The distended ampulla of oviduct was found under a dissecting microscope. By tearing it open with thin pointed forceps, the eggs with cumulus cells flowed out. The fertilized eggs with cumulus cells were then transferred into M2 culture medium containing 1 mg/ml hyaluronidase to lyse the cumulus cells, and the eggs were immediately pipetted into fresh M2 culture medium with a previously pulled pipette to wash 2-3 times and then pipetted into another fresh M2 culture medium. This was done 4-5 times until there were no tissue debris and blood cells in the field of view. Finally, the fertilized eggs were pipetted into the drop (about 50 μl) of M16 culture medium which had been balanced with CO₂, and then the drop was covered with a layer of embryo-grade paraffin oil and placed in the incubator for culture. After the pronucleus of eggs was clearly and fully developed, microinjection could be performed. A capillary glass tube with an outer diameter of 1 mm and an inner diameter of 0.75-0.8 mm was selected and installed on a horizontal micropipette puller to draw an injection needle and egg-holding needle.

A drop of about 50 μl of M2 culture medium was added in the center of 35 cm culture dish, and 5 ml of liquid paraffin oil was added. The culture medium drop was adjusted to be at the center of microscope field. Then 10-20 fertilized eggs with clear nucleus were aspirated and add to the center of the drop. The syringe of the egg-holding needle was adjusted to form negative pressure in the egg-holding needle to aspirate the fertilized eggs with clear edge, full shape and obvious nucleus, and the position of the fertilized eggs were adjusted by changing the pressure of the syringe, so that the male nucleus of the fertilized egg was directly opposite to the injection needle tip with the minimum distance between the two. The positions of the injection needle and egg-holding needle were adjusted, so that the nucleus and injection needle tip had the best clarity. The lever was pushed to carefully inject so that the needle tip entered the nucleus. The injection pressure was increased until the nucleus apparently swollen. The needle was pulled out gently and quickly until it left the cell. Cas9 mRNA was injected in an amount of 100 ng/μl, gRNA in an amount of 20 ng/μl and Or35a-P2A-Or83b-P2 A donor mRNA in an amount of 100 ng/μl. After injection, the fertilized eggs were returned to M16 culture medium and cultured overnight in a cell culture incubator. On the next day, the two-cell embryo was transferred from M16 culture medium to M2 culture medium with embryo transfer needle and washed once or twice. A new transfer tube was used to inject a small amount of paraffin oil and a little M2 culture medium, and then 10-15 injected two-cell embryos were aspirated to prepare for transplantation.

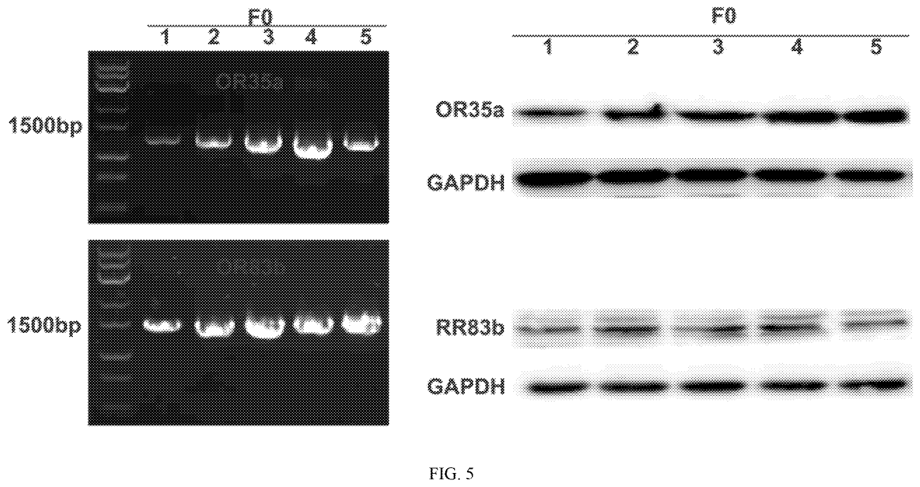

Female ICR mice (over 6 weeks old) in estrus were selected to be cohabited with male ligated mice, and those with vaginal plug were selected on the next day to be surrogate pregnant mice. After anesthetization, the surrogate mice were clipped free of fur on the back with iris tissue scissors and disinfected with 75% alcohol. A longitudinal incision about 1 cm long was cut at 1 cm beside the intersection of the posterior median line and the horizontal line of the flat costal margin, and the oviduct was pulled out with forceps. The oviduct and ovarian capsule were carefully torn using microscopic forceps. After the opening of oviduct was found, the embryo transfer tube was quickly inserted to inject the embryo for embryo transplantation. Pseudopregnant mice were incubated and allowed to recover. After parturition, the F₀ generation mice were obtained.
5. The obtained F₀ generation mice were detected preliminarily by PCR and sequencing, and then the target sites may be further sampled for mRNA determination of transcription level and protein determination of translation level. As shown in FIG. 5, tissues from the F0 generation mouse were taken for PCR and Western blot identification, suggesting that the mouse tissues contained the gene of interest.

### Test Example 1. Verification of the Arrival of 2-Pentanone in Cerebrospinal Fluid

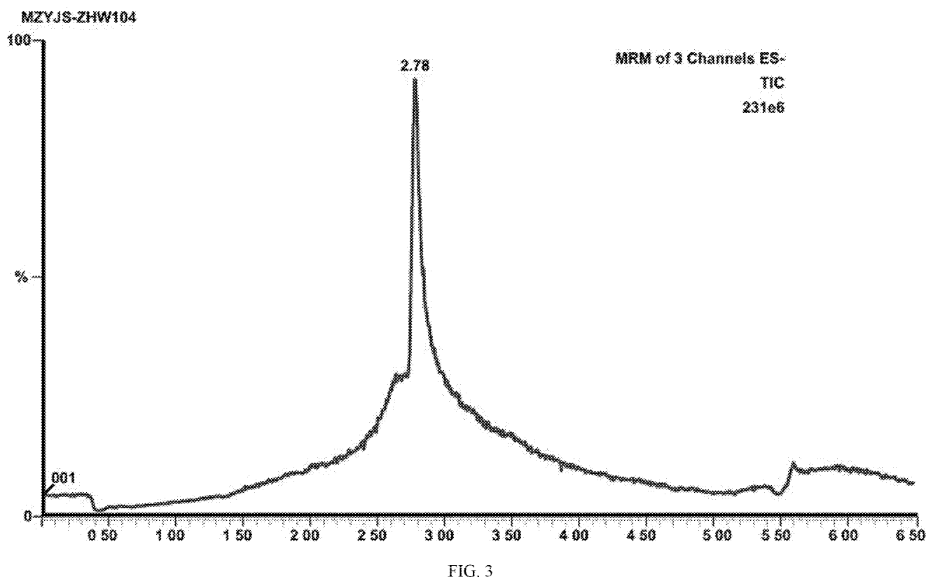

The specific method for detecting the arrival of 2-pentanone in cerebrospinal fluid in this test example included the following: rats were given 2-pentanone for 3 min, then blood and cerebrospinal fluid were drawn, immediately sealed and centrifuged at high speed to pellet red blood cells. The obtained cerebrospinal fluid and blood samples were subjected to HPLC-MS detection. Prior to detection, 100 μL of samples was taken and added with 400 μL of acetonitrile. The protein was separated by vortex and centrifuged at 14000 rpm for 10 min, and 100 μL of supernatant was used for HPLC-MS detection. FIG. 3 shows the results of HPLC-MS detection of 2-pentanone, suggesting that the peak time was 2.72 min. It was confirmed by high performance liquid chromatography-mass spectrometry (HPLC-MS) that 2-pentanone could reach cerebrospinal fluid through blood rapidly (3 min) after inhaled by animals, and the concentration in cerebrospinal fluid decreased rapidly within 3-5 min after the inhalation was stopped.

### Test Example 2. Effect of 2-Pentanone on Cellular Calcium Content and Neuronal Firing Rate

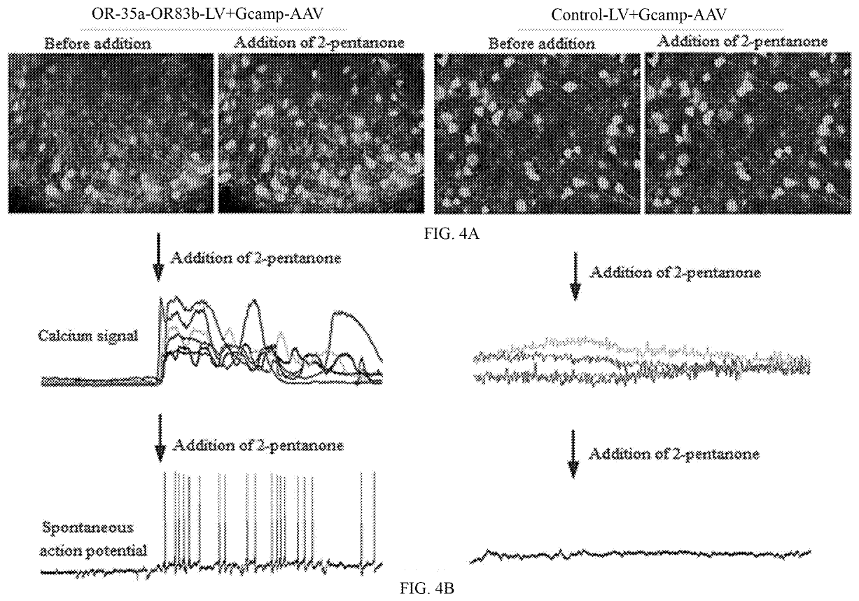

After the specific receptor of 2-pentanone according to the present disclosure was expressed in cells, the effects of 2-pentanone on cellular calcium content and neuronal firing rate were demonstrated by the calcium imaging experiment and patch clamp experiment.
1. Calcium imaging experiment: Adult C57 mice were anesthetized, fixed on a stereotaxic apparatus, and operated to expose the brain surface of the mice (1.5 mm behind the bregma, 1 mm aside, and injection depth of 2 mm). The overexpression virus (1 μl) constructed in Example 1 and the calcium imaging virus (0.5 μl) were simultaneously injected into the mouse brain using a microinjector. After 2 weeks of feeding, the brains of mice were removed. The brain tissues near the injection site were cut into 150 μm tissue slices using a vibrating microtome and placed in artificial cerebrospinal fluid with a mixture of 5% CO₂ and 95% O₂, and calcium imaging experiment was performed using a confocal microscope. As shown in FIG. 4A, after co-expression of the specific receptor of 2-pentanone in cells and tissues, it was confirmed by calcium imaging experiment that the addition of 2-pentanone resulted in a significantly increased intracellular calcium ion concentration.

1. A method for regulating a cell, comprising expressing a specific receptor of 2-pentanone in a target cell of the specific receptor, wherein the target cell is regulated by a binding of the 2-pentanone to the specific receptor of the 2-pentanone to activate a cyclic ligand-gated cation channel and result in the increase in the intracellular calcium ion concentration, a depolarization of cell membranes, and a generation of an electrical activity or a corresponding physiological activity.

2. The method according to claim 1, wherein the specific receptor of the 2-pentanone is expressed in the target cell via a recombinant expression vector or a recombinant cell, and the recombinant expression vector or the recombinant cell comprises a nucleic acid molecule encoding the 2-pentanone.

3. The method according to claim 1, wherein the specific receptor of the 2-pentanone comprises Or35a having an amino acid sequence set forth in SEQ ID NO: 4 and Or83b having an amino acid sequence set forth in SEQ ID NO: 5.
